(19) United States
(12) Patent Application Publication
(10) Pub. No.: US 2011/0150862 A1
(43) Pub. Date: Jun. 23, 2011

(54) INHIBITORS OF STIM1 FOR THE TREATMENT OF CARDIOVASCULAR DISORDERS

(76) Inventors: Jean-Sébastien Hulot, Paris (FR); Anne-Marie Lompre, Paris (FR)

(21) Appl. No.: 12/936,951

(22) PCT Filed: Apr. 8, 2009
(86) PCT No.: PCT/EP09/54237

§ 371 Date: Feb. 28, 2011

(51) Int. Cl. A61K 39/395; A61K 31/713; A61K 31/7088; A61P 9/00

(52) U.S. Cl. 424/130.1; 514/44 A; 514/44 R

(57) ABSTRACT

The invention relates to inhibitors of STIM1 for the treatment and/or the prevention of cardiac disorders such as atherosclerosis, post-angioplasty restenosis, pulmonary arterial hypertension, vein-graft disease, cardiac hypertrophy, cardiac arrhythmias, valvulopathies, diastolic dysfunction, chronic heart failure, ischemic heart failure, and myocarditis.

### FIELD OF THE INVENTION

The invention relates to inhibitors of Stromal Interaction Molecule 1 (STIM1) for the treatment and/or the prevention of cardiac disorders, such as cardiac hypertrophy and heart failure and for vascular disorders such as atherosclerosis, post-angioplasty restenosis, pulmonary arterial hypertension and vein-graft disease. The present invention concerns gene regulation and cellular physiology in cardiomyocytes and smooth muscle cells.

### BACKGROUND OF THE INVENTION

Cellular proliferation and growth are two mechanisms leading to cardiovascular remodelling commonly observed in vascular and cardiac muscular cells in response to diverse pathological stimuli. Excessive smooth muscle cells proliferation is a fundamental process that contributes to the injury response in major arterial vessels. Such process is involved in numerous vascular disorders including atherosclerosis, post-angioplasty restenosis, pulmonary arterial hypertension and vein-graft disease (Dzau V J and al., 2002; Novak K., 1998). Identifying modifiers of vascular smooth muscle cell (VSMC) proliferation is thus a major focus of research in cardiovascular biology and medicine.

On the other hand, hypertrophic cardiac remodelling is an adaptive response of the heart to many forms of cardiac disease, including hypertension, mechanical load abnormalities, myocardial infarction, valvular dysfunction, cardiac arrhythmias, endocrine disorders and genetic mutations in cardiac contractile protein genes. For a wide time, the hypertrophic response of cardiomyocytes has been considered as a useful compensatory state to maintain cardiac performance. However, it is now considered that such remodelling following disease-inducing stimuli is maladaptive and contributes to heart failure progression and favour arrhythmia and sudden death. Accordingly, cardiac hypertrophy has been established as an independent risk factor for cardiac morbidity and mortality.

In both cases, stereotypical pattern of changes in gene expression that include the re-expression of fetal genes are observed. Such differences are controlled by particular underlying signalling pathways. For example, it has been shown that acquisition of proliferating phenotype by VSMC is associated with major alterations in Ca2+ handling. Modulations in Ca2+ signal alter gene expression by activating different kinases, phosphatases, and Ca2+-regulated transcription factors such as NFAT (nuclear factor of activated T lymphocytes). Recently, it has been shown that that increasing the rate of sarcoplasmic reticular (SR) calcium uptake by restoring sarcoplasmic reticulum Ca2+ ATPase (SERCA2a) expression inhibit VSMC proliferation and prevent neointima formation induced by injury (Lipskaia L et al. 2005). Accordingly it has been suggested that restenosis can be treated by administering an agent that increases SERCA activity (e.g. WO2005023292).

### SUMMARY OF THE INVENTION

The invention relates to an inhibitor of STIM1 for inhibiting the growth and proliferation of smooth muscle cells and/or the hypertrophic response of cardiomyocytes.

The invention relates to an inhibitor of STIM1 for the treatment of a cardio-vascular disorder. Examples of vascular disorders which may be treated with STIM1 inhibitors are atherosclerosis, post-angioplasty restenosis, pulmonary arterial hypertension and vein-graft disease. Examples of cardiac disorders which may be treated with STIM1 inhibitors are cardiac hypertrophy and heart failure following diverse pathological stimuli such as hypertension, myocardial infarction and ischemic cardiopathies or coronary artery diseases, cardiac arrhythmias, mechanical over-load, toxic origin, endocrine disorders, and genetic mutations in cardiac contractile protein genes.

The invention relates to a method for treating a cardio-vascular disorder in a subject comprising administering to the subject a therapeutically effective amount of a pharmaceutical composition comprising an inhibitor of STIM1.

The invention also relates to the use of an inhibitor of STIM1 for the manufacture of a medicament for inhibiting the proliferation of smooth muscle cells and/or the growth of cardiomyocytes.

### DETAILED DESCRIPTION OF THE INVENTION

The instant application formally demonstrates for the first time that smooth muscle cells proliferation may be inhibited by inhibiting STIM1. It also demonstrates for the first time that STIM1 is present in the cardiomyocyte and that inhibiting STIM1 expression prevents cardiomyocyte growth in vitro.

### DEFINITIONS

The term “STIM1” has its general meaning in the art and refers to Stromal Interaction Molecule 1. The term may include naturally occurring STIM1s and variants and modified forms thereof. The term may also refer to fusion proteins in which a domain from STIM1 that retains at least one STIM1 activity is fused, for example, to another polypeptide (e.g., a polypeptide tag such as are conventional in the art). The STIM1 can be from any source, but typically is a mammalian (e.g., human and non-human primate) STIM1, particularly a human STIM1. An exemplary native STIM1 amino acid sequence is provided in GenPept database under accession number AAH21300 and an exemplary native nucleotide sequence encoding for STIM1 is provided in GenBank database under accession number NM_{—}003156.

The expression “inhibitor of STIM1” should be understood broadly, it encompasses inhibitors of the STIM1 mediated cellular efflux of Ca2+, hereafter called STIM1 activity, and inhibitors of the expression of STIM1.

An “inhibitor of expression” refers to a natural or synthetic compound that has a biological effect to inhibit or significantly reduce the expression of a gene. Consequently an “inhibitor of STIM1 expression” refers to a natural or synthetic compound that has a biological effect to inhibit or significantly reduce the expression of the gene encoding for the STIM1 gene.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

As used herein, the term “subject” denotes a mammal, such as a rodent, a feline, a canine, and a primate. Preferably, a subject according to the invention is a human.

In its broadest meaning, the term “treating” or “treatment” refers to reversing, alleviating, inhibiting the progress of, or preventing the disorder or condition to which such term applies, or one or more symptoms of such disorder or condition.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

By “biocompatible” is meant a material which elicits no or minimal negative tissue reaction including e.g. thrombus formation and/or inflammation.

### Therapeutic Methods and Uses

The present invention provides methods and compositions (such as pharmaceutical compositions) for inhibiting the proliferation of smooth muscle cells, in particular arterial smooth muscle cells. The present invention also provides methods and compositions (such as pharmaceutical compositions) for treating and/or preventing vascular disorders such as atherosclerosis, post-angioplasty restenosis, pulmonary arterial hypertension and vein-graft disease. The present invention also provides methods and compositions (such as pharmaceutical compositions) for inhibiting the hypertrophic response of cardiomyocytes. The present invention also provides methods and compositions (such as pharmaceutical compositions) for treating and/or preventing cardiac hypertrophy cardiac arrhythmias, valvulopathies, diastolic dysfunction, chronic heart failure, ischemic heart failure, and myocarditis. The treatment may improve one or more symptoms of cardiac hypertrophy or heart failure, such as providing increased exercise capacity, increased blood ejection volume, left ventricular end diastolic pressure, left ventricular end systolic and diastolic dimensions, wall tension and wall thickness, quality of life, disease-related morbidity and mortality, reversal of progressive remodeling, improvement of ventricular dilation, increased cardiac output, relief of impaired pump performance, improvement in arrhythmia.

Thus, an object of the invention is an inhibitor of STIM1 for inhibiting the proliferation of smooth muscle cells or for inhibiting the hypertrophic response of cardiomyocyte. The inhibitor of STIM1 may be used (1) for the treatment and/or the prevention of vascular disorders such as atherosclerosis, post-angioplasty restenosis, and pulmonary arterial hypertension vein-graft disease, (2) for treating and/or preventing cardiac hypertrophy or heart failure

In one embodiment, the STIM1 inhibitor may be a low molecular weight inhibitor, e.g. a small organic molecule.

In another embodiment the STIM1 inhibitor is an antibody or antibody fragment that can partially or completely block the STIM1 transport activity (i.e. a partial or complete STIM1 blocking antibody or antibody fragment).

In particular, the STIM1 inhibitor may consist in an antibody directed against the STIM1, in such a way that said antibody blocks the activity of STIM1.

Antibodies directed against the STIM1 can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies against STIM1 can be prepared and isolated using any technique that provides for the production of antibody molecules by continuous cell lines in culture. Techniques for production and isolation include but are not limited to the hybridoma technique originally described by Kohler and Milstein (1975); the human B-cell hybridoma technique (Cote et al., 1983); and the EBV-hybridoma technique (Cole et al. 1985). Alternatively, techniques described for the production of single chain antibodies (see, e.g., U.S. Pat. No. 4,946,778) can be adapted to produce anti-STIM1, single chain antibodies. STIM1 inhibitors useful in practicing the present invention also include anti-STIM1 fragments including but not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab and/or scFv expression libraries can be constructed to allow rapid identification of fragments having the desired specificity to STIM1.

Humanized anti-STIM1 antibodies and antibody fragments thereof can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. Methods for making humanized antibodies are described, for example, by Winter (U.S. Pat. No. 5,225,539) and Boss (Celltech, U.S. Pat. No. 4,816,397).

In still another embodiment, the inhibitor of STIM1 is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Another aspect of the invention relates to selective inhibitor of STIM1 expression.

Inhibitors of STIM1 expression for use in the present invention may be based on anti-sense oligonucleotide constructs. Anti-sense oligonucleotides, including anti-sense RNA molecules and anti-sense DNA molecules, would act to directly block the translation of STIM1 mRNA by binding thereto and thus preventing protein translation or increasing mRNA degradation, thus decreasing the level of STIM1s, and thus activity, in a cell. For example, antisense oligonucleotides of at least about 15 bases and complementary to unique regions of the mRNA transcript sequence encoding STIM1 can be synthesized, e.g., by conventional phosphodiester techniques and administered by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Small inhibitory RNAs (siRNAs) can also function as inhibitors of STIM1 expression for use in the present invention. STIM1 expression can be reduced by contacting a subject or cell with a small double stranded RNA (dsRNA), or a vector or construct causing the production of a small double stranded RNA, such that STIM1 expression is specifically inhibited (i.e. RNA interference or RNAi). Methods for selecting an appropriate dsRNA or dsRNA-encoding vector are well known in the art for genes whose sequence is known (e.g. see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and International Patent Publication Nos. WO 01/36646, WO 99/32619, and WO 01/68836). A siRNA efficiently silencing STIM1 has been developed. The sense sequence is 5′-GGGAAGACCUCAAUUACCAdtdt-3′ (SEQ ID NO:1) and anti-sense: 5′-UGGUAAUUGAGGUCUUCCCdtdt-3′ (SEQ ID NO:2).

shRNAs (short hairpin RNA) can also function as inhibitors of STIM1 expression for use in the present invention. An example of short hairpin RNA according to the invention is a shRNA comprising a sense sequence 5′-GGGAAGACCTCAATTACCA-3′ (SEQ ID NO:3) and an anti-sense sequence 5′-TGGTAATTGAGGTCTTCCC-3′ (SEQ ID NO:4).

Ribozymes can also function as inhibitors of STIM1 expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of STIM1 mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable.

Both antisense oligonucleotides and ribozymes useful as inhibitors of STIM1 expression can be prepared by known methods. These include techniques for chemical synthesis such as, e.g., by solid phase phosphorothioate chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a mean of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and preferably cells expressing STIM1. Preferably, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

Preferred viral vectors are based on non-cytopathic eukaryotic viruses in which non-essential genes have been replaced with the gene of interest. Non-cytopathic viruses include retroviruses (e.g., lentivirus), the life cycle of which involves reverse transcription of genomic viral RNA into DNA with subsequent proviral integration into host cellular DNA. Retroviruses have been approved for human gene therapy trials. Most useful are those retroviruses that are replication-deficient (i.e., capable of directing synthesis of the desired proteins, but incapable of manufacturing an infectious particle). Such genetically altered retroviral expression vectors have general utility for the high-efficiency transduction of genes in vivo. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the target cells with viral particles) are provided in Kriegler, 1990 and in Murry, 1991).

Preferred viruses for certain applications are the adenoviruses and adeno-associated (AAV) viruses, which are double-stranded DNA viruses that have already been approved for human use in gene therapy. Actually 12 different AAV serotypes (AAV1 to 12) are known, each with different tissue tropisms (Wu, Z Mol Ther 2006; 14:316-27). Recombinant AAV are derived from the dependent parvovirus AAV2 (Choi, V W J Virol 2005; 79:6801-07). The adeno-associated virus type 1 to 12 can be engineered to be replication deficient and is capable of infecting a wide range of cell types and species (Wu, Z Mol Ther 2006; 14:316-27). It further has advantages such as, heat and lipid solvent stability; high transduction frequencies in cells of diverse lineages, including hemopoietic cells; and lack of superinfection inhibition thus allowing multiple series of transductions. Reportedly, the adeno-associated virus can integrate into human cellular DNA in a site-specific manner, thereby minimizing the possibility of insertional mutagenesis and variability of inserted gene expression characteristic of retroviral infection. In addition, wild-type adeno-associated virus infections have been followed in tissue culture for greater than 100 passages in the absence of selective pressure, implying that the adeno-associated virus genomic integration is a relatively stable event. The adeno-associated virus can also function in an extrachromosomal fashion and most recombinant adenovirus are extrachromosomal.

Other vectors include plasmid vectors. Plasmid vectors have been extensively described in the art and are well known to those of skill in the art. See e.g. Sambrook et al., 1989. In the last few years, plasmid vectors have been used as DNA vaccines for delivering antigen-encoding genes to cells in vivo. They are particularly advantageous for this because they do not have the same safety concerns as with many of the viral vectors. These plasmids, however, having a promoter compatible with the host cell, can express a peptide from a gene operatively encoded within the plasmid. Some commonly used plasmids include pBR322, pUC18, pUC19, pRC/CMV, SV40, and pBlueScript, pSIREN. Other plasmids are well known to those of ordinary skill in the art. Additionally, plasmids may be custom designed using restriction enzymes and ligation reactions to remove and add specific fragments of DNA. Plasmids may be delivered by a variety of parental, mucosal and topical routes. For example, the DNA plasmid can be injected by intramuscular, intradermal, subcutaneous, or other routes. It may also be administered by intranasal sprays or drops, rectal suppository and orally. It may also be administered into the epidermis or a mucosal surface using a gene-gun. The plasmids may be given in an aqueous solution, dried onto gold particles or in association with another DNA delivery system including but not limited to liposomes, dendrimers, cochleate and microencapsulation.

In a preferred embodiment, the antisense oligonucleotide, siRNA, shRNA or ribozyme nucleic acid sequence is under the control of a heterologous regulatory region, e.g., a heterologous promoter. The promoter can be, e.g., a smooth muscle specific promoter, such as a smooth muscle alpha actin promoter, SM22a promoter, cardiac specific promoter, such as cardiac myosin promoter (e.g., a cardiac myosin light chain 2v promoter), troponin T promoter, or BNP promoter. The promoter can also be, e.g., a viral promoter, such as CMV promoter or any synthetic promoters.

The selective inhibitor of STIM1 activity and/or expression may be administered in the form of a pharmaceutical composition, as defined below.

Preferably, said inhibitor is administered in a therapeutically effective amount.

By a “therapeutically effective amount” is meant a sufficient amount of the STIM1 inhibitor to treat and/or to prevent vascular disorders at a reasonable benefit/risk ratio applicable to any medical treatment.

It will be understood that the total daily usage of the compounds and compositions of the present invention will be decided by the attending physician within the scope of sound medical judgment. The specific therapeutically effective dose level for any particular patient will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

### Screening Methods

Inhibitors of the invention can be further identified by screening methods described in the state of the art. The screening methods of the invention can be carried out according to known methods.

The screening method may measure the binding of a candidate compound to STIM1, or to cells or membranes bearing STIM1, or a fusion protein thereof by means of a label directly or indirectly associated with the candidate compound. Alternatively, a screening method may involve measuring or, qualitatively or quantitatively, detecting the competition of binding of a candidate compound to the receptor with a labelled competitor (e.g., inhibitor or substrate).

For example, STIM1 cDNA may be inserted into an expression vector that contains necessary elements for the transcription and translation of the inserted coding sequence. Following vector/host systems may be utilized such as Baculovirus/Sf9 Insect Cells Retrovirus/Mammalian cell lines like HepB3, LLC-PK1, MDCKII, CHO, HEK293 Expression vector/Mammalian cell lines like HepB3, LLC-PK1, MDCKII, CHO, HEK293. Such vectors may be then used to transfect cells so that said cells express recombinant STIM1 at their membrane. It is also possible to use cell lines expressing endogenous STIM1 protein (THP-1, U937, WI-38, WI-38 (VA-13 subline), IMR-90, HEK-293).

Cells obtained as above described may be the pre-incubated with test compounds and subsequently stimulated with compounds known to elevate cellular Ca2+ efflux (such as). Test compounds are screened for their ability to inhibit intracellular Ca2+ levels.

### Pharmaceutical Compositions

A further object of the invention relates to a pharmaceutical composition for treating and/or preventing vascular disorders such as atherosclerosis, post-angioplasty restenosis, and pulmonary arterial hypertension vein-graft disease and for treating and/or preventing cardiac hypertrophy or heart failure said composition comprising a selective inhibitor of STIM1 expression and/or activity

The STIM1 inhibitor may be combined with pharmaceutically acceptable excipients, and optionally sustained-release matrices, such as biodegradable polymers, to form therapeutic compositions.

In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The STIM1 inhibitor of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active polypeptides in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

The STIM1 inhibitor of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds of the invention formulated for parenteral administration, such as intravenous or intramuscular injection, other pharmaceutically acceptable forms include, e.g. tablets or other solids for oral administration; liposomal formulations; time release capsules; and any other form currently used.

Pharmaceutical compositions of the inventions may include any other anti-proliferative agent that reduces smooth muscle cell proliferation. For example, the anti-proliferative agent may be rapamycin, rapamycin derivatives, paclitaxel, docetaxel, 40-0-(3-hydroxy)propyl-rapamycin, 40-O-[2-(2-hydroxy)ethoxy]ethyl-rapamycin, and 40-O-tetrazole-rapamycin, ABT-578, everolimus and combinations thereof. Pharmaceutical compositions may also include phosphodiesterase (PDE) inhibitors as those described in documents US2005234238 DE10156229, DE10135009, WO0146151, WO2005012303 and US2006106039. More particularly, pharmaceutical compositions of the invention may comprise any further agent that increases SERCA activity as those described in document WO2005023292.

### Biomaterials

The present invention also relates to the use of an inhibitor of STIM1 for the preparation of biomaterials or medical delivery devices selected among endovascular prostheses, such as stents, bypass grafts, internal patches around the vascular tube, external patches around the vascular tube, vascular cuff, and angioplasty catheter.

In this respect, the invention relates more particularly to biomaterials or medical delivery devices as mentioned above, coated with such inhibitor of STIM1 expression and/or activity as defined above, said biomaterials or medical devices being selected among endovascular prostheses, such as stents, bypass grafts, internal patches around the vascular tube, external patches around the vascular tube, vascular cuff, and angioplasty catheter. Such a local biomaterial or medical delivery device can be used to reduce stenosis or restenosis as an adjunct to revascularization, bypass or grafting procedures performed in any vascular location including coronary arteries, carotid arteries, renal arteries, peripheral arteries, cerebral arteries or any other arterial or venous location, to reduce anastomic stenosis such as in the case of arterial-venous dialysis access with or without polytetrafluoro-ethylene grafting and with or without stenting, or in conjunction with any other heart or transplantation procedures, or congenital vascular interventions.

For illustration purpose, such endovascular prostheses and methods for coating selective inhibitor thereto are more particularly described in WO2005094916, or are those currently used in the art. The compounds used for the coating of the prostheses should preferentially permit a controlled release of said inhibitor. Said compounds could be polymers (such as sutures, polycarbonate, Hydron, and Elvax), biopolymers/biomatrices (such as alginate, fucans, collagen-based matrices, heparan sulfate) or synthetic compounds such as synthetic heparan sulfate-like molecules or combinations thereof (Davies, et al., 1997; Desgranges, et al., 2001; Dixit, et al., 2001; Ishihara, et al., 2001; Letourneur, et al., 2002; Tanihara, et al., 2001; Tassiopoulos and Greisler, 2000). Other examples of polymeric materials may include biocompatible degradable materials, e.g. lactone-based polyesters or copolyesters, e.g. polylactide; polylactide-glycolide; polycaprolactone-glycolide; polyorthoesters; polyanhydrides; polyaminoacids; polysaccharides; polyphosphazenes; poly (ether-ester) copolymers, e.g. PEO-PLLA, or mixtures thereof; and biocompatible non-degrading materials, e.g. polydimethylsiloxane; poly (ethylene-vinylacetate); acrylate based polymers or copolymers, e.g. polybutylmethacrylate, poly (hydroxyethyl methylmethacrylate); polyvinyl pyrrolidinone; fluorinated polymers such as polytetrafluoethylene; cellulose esters. When a polymeric matrix is used, it may comprise 2 layers, e.g. a base layer in which said inhibitor is incorporated, such as ethylene-co-vinylacetate and polybutylmethacrylate, and a top coat, such as polybutylmethacrylate, which acts as a diffusion-control of said inhibitor. Alternatively, said inhibitor may be comprised in the base layer and the adjunct may be incorporated in the outlayer, or vice versa.

Such biomaterial or medical delivery device may be biodegradable or may be made of metal or alloy, e.g. Ni and Ti, or another stable substance when intended for permanent use. The inhibitor of the invention may also be entrapped into the metal of the stent or graft body which has been modified to contain micropores or channels. Also internal patches around the vascular tube, external patches around the vascular tube, or vascular cuff made of polymer or other biocompatible materials as disclosed above that contain the inhibitor of the invention may also be used for local delivery.

Said biomaterial or medical delivery device allow the inhibitor releasing from said biomaterial or medical delivery device over time and entering the surrounding tissue. Said releasing may occur during 1 month to 1 year. The local delivery according to the present invention allows for high concentration of the inhibitor of the invention at the disease site with low concentration of circulating compound. The amount of said inhibitor used for such local delivery applications will vary depending on the compounds used, the condition to be treated and the desired effect. For purposes of the invention, a therapeutically effective amount will be administered.

The local administration of said biomaterial or medical delivery device preferably takes place at or near the vascular lesions sites. The administration may be by one or more of the following routes: via catheter or other intravascular delivery system, intranasally, intrabronchially, interperitoneally or eosophagal. Stents are commonly used as a tubular structure left inside the lumen of a duct to relieve an obstruction. They may be inserted into the duct lumen in a non-expanded form and are then expanded autonomously (self-expanding stents) or with the aid of a second device in situ, e.g. a catheter-mounted angioplasty balloon which is inflated within the stenosed vessel or body passageway in order to shear and disrupt the obstructions associated with the wall components of the vessel and to obtain an enlarged lumen.

The invention will be further illustrated by the following figures and examples. However, these examples and figures should not be interpreted in any way as limiting the scope of the present invention.

### FIGURES

FIG. 1. STIM1 is expressed in vascular smooth muscle cells. A—Western-blots of total extracts from human coronary artery, human and rat vascular smooth muscle cells and of Jurkat cells hybridized with anti-GOK/Stim. B—Confocal imaging of hCASMC labelled with anti-STIM1 and anti-SERCA2 (IID8).

FIG. 2. STIM1 is upregulated in proliferative VSMC. A—Relative STIM1 mRNA levels normalized to RPL32 mRNA in quiescent (0.1% S) and proliferative (5% S) hCASMC. B—Western-blot showing expression of STIM1, calcineurin (PP2B) and cyclin D1 according to conditions. C—STIM1 (grey bars) and cyclin D1 (black bars) protein levels normalized to PP2B level in quiescent and proliferative hCASMC. **p<0.01; ***p<0.001.

FIG. 3. STIM1 knockdown inhibits hCASMC proliferation in vitro. A—Western-blot showing the disappearance of STIM 1 and the reduction of cyclin D1 expression 72 hours after transfection with STIM1 siRNA compared to the negative control (scrambled) siRNA. B—Proliferation (measured by BrDU incorporation) of hCASMC in presence of 5% supplement mix or C-50 nM PDGF-BB in control cells or cells transfected with STIM1 or scrambled siRNA for 72 hours, or treated with 5 μM cyclosporin A (CsA) for 24 h. *p<0.05; **p<0.01

FIG. 4. Adenoviral vector expressing specific STIM1 shRNA prevents in vivo neointima formation in rat injured carotid artery. A—Sequence of STIM1 shRNA. B—Average intima/media thickness ratios for the above three groups (***p<0.001 compared with Ad-shLuc). M indicates media; ni, neointima; and ad, adventitia (n=5 for non-injured carotid, n=4 for Ad-shLuc and n=6 for Ad-shSTIM1). C—PCR analysis of DNA extracted from the vessels.

FIG. 5. STIM1 silencing inhibits TRPC currents. Representative single channel activity records obtained from cell-attached membranes on scrambled siRNA-transfected cells (top) and STIM1 siRNA-transfected cells (bottom) at a holding potential of −80 mV. In a and b are presented an expansion of the channel activity.

FIG. 6. RNA interference-induced STIM1 silencing prevents NFAT nuclear translocation and activity and enhances CREB activity. A—Measurement of NFAT activity using a NFAT-driven luciferase construct in control cells. B—Measurement of the relative MCIP mRNA level normalized to RPL32 mRNA. Cyclosporin A (CsA) is used as a negative control. C—Measurement of cAMP responsive element (CRE) activity using a CRE-driven luciferase construct

FIG. 7: STIM1 expression in total hearts samples and in isolated cardiomyocytes. A—RT-PCR experiments showing STIM1 mRNA expression in human hearts samples (left and right atria as well as left ventricule). B—Western blotting experiments on total proteins extracts from human and rat left ventricules. A 90-KDa band is identified as found in the Jurkat T cells (a classical model for STIM1 expression). Analysis of STIM1 expression in isolated neonatal rat cardiomyocyte shows STIM1 expression at isolation (J0) and during the 7 following days while cardiomyocytes are cultured. C—Western blotting experiments on total proteins extracts of adult rats isolated ventricule cardiomyocytes (left panel) or other cardiac cells (fibroblasts, immune cells, smooth muscle cells, right panel). STIM1 expression is observed in both cellular cell types.

FIG. 8. STIM1 is over-expressed in an animal model of cardiac hypertrophy (Abdominal aortic banding vs SHAM). Five weeks after banding rats (n=6/groups) are sacrified and evaluated. A—(A) Morphologic characteristics of rats (heart weight and body weight) showing the increased heart weight in banding rats and (B) Echocardiographic and haemodynamic assessment showing a significant cardiac hypertrophy and a significant increase in arterial pressure. B—STIM1 mRNA (normalized to RPL32 mRNA) in hearts from banding rats vs SHAM. Markers of cardiac failure (ANF and MCIP1) are also significantly increased. C—Western-blotting showing STIM1 over-expression in banding rats compared to SHAM. D—STIM1 protein level normalized to PP2B level is significantly increased and correlated to heart weight/body weight ratio.

FIG. 9 STIM1 is upregulated in hypertrophic cardiomyocytes. Isolated rat cardiomyocytes were stimulated with phenylephrine (50 μM) or Endothelin 1 for 48 h. A—Typical imaging of non stimulated cardiomyocytes and hypertrophic cardiomyocytes (induced by phenylephrine or endothelin 1) labelled with alpha-actinin. A significant increase in cardiomyocyte area is observed. B—STIM1 mRNA and ANF mRNA (normalized to RPL32 mRNA) in non-stimulated and stimulated cardiomyocytes. C—STIM1 protein levels (normalized to PP2B/Calcineurin) in stimulated compared to non-stimulated cardiomyocytes

FIG. 10. Efficiency of adenoviral vector encoding for short hairpin RNA against STIM1 mRNA to silence STIM1 expression. A—Dose-relationship on STIM1 mRNA level (normalized to PRL32 mRNA). B—Western blotting and protein level analysis showing decreased STIM1 expression in isolated rat cardiomyocytes infected with ad-ShSTIM1 compared to ad-shLuciferase (negative control).

FIG. 11. STIM1 knockdown prevents cardiac hypertrophy in vitro. A—Typical pattern of isolated cardiomyocyte stimulated for 48 hours with phenylephrine and infected with either adv-ShLuciferase or Adv-ShSTIM1 showing a significant reduction in cardiomyocyte surface area. B—STIM1 knockdown inhibited neonatal cardiomyocytes protein synthesis on in vitro. 3H-leucine incorporation was measured in uninfected neonatal cardiomyocytes (control) or myocytes infected with Ad.shRNA STIM-1 for 72 hours or negative control (scrambled) Ad.shRNA. Phenylephrine stimulation (50 m) was applied for 48 hours. The mean values±SEM are shown. C— Analysis of cardiomyocyte surface area (3 experiments, with analysis of 50 cells/conditions for each experiment) in PE-stimulated non-infected neonatal cardiomyocytes or myocytes infected with Ad.shRNA STIM-1 for 72 hours or negative control (scrambled) Ad.shRNA. D—& E—. Quantitative real-time PCR showing ANF (D) and MCIP1 (E) down-expression in isolated cardiomyocyte infected with Adv-shSTIM1 compared to those infected with the scrambled Ad-ShRNA.

### EXAMPLE 1

### STIM1 and Vascular Smooth Muscle Cell (VSMC) Proliferation

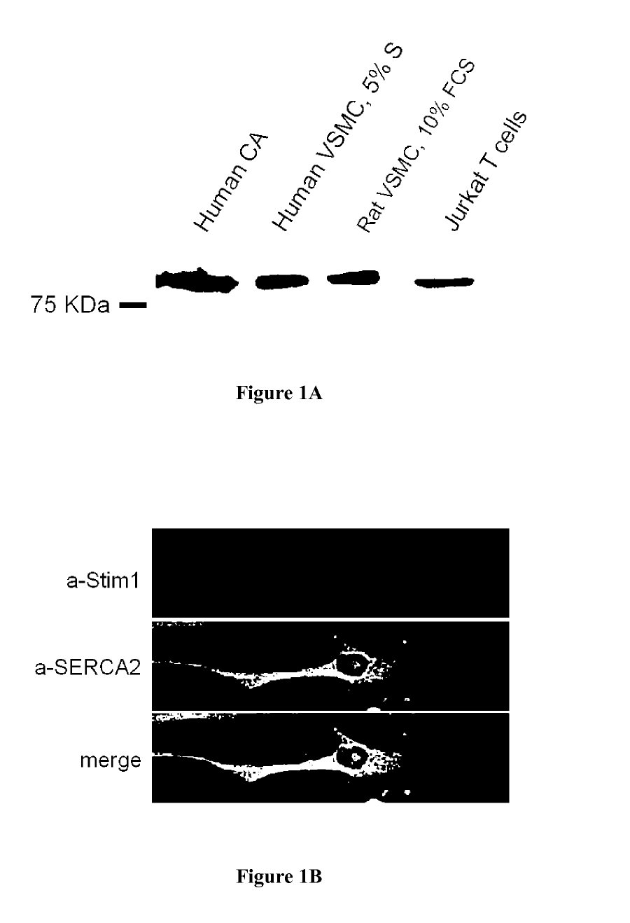

STIM1 is expressed in vascular smooth muscle cells: Immunofluorescence analysis of balloon-injured rat carotid arteries (a well-characterized model of SMC proliferation) revealed that STIM 1 was expressed in the media as well as in highly proliferative SMC in the neointima. The expected 90 kDa protein (the same molecular weight than the protein observed in human Jurkat T cell) was present in both vascular smooth muscle cells isolated from human coronary artery (hCASMC) and in rat aorta smooth muscle cells (FIG. 1A). Confocal immunofluorescence analysis in isolated vascular smooth muscle cells revealed a predominant endoplasmic reticulum distribution of STIM1, which was similar to the one of SERCA2, an endoplasmic reticulum marker (FIG. 1B).

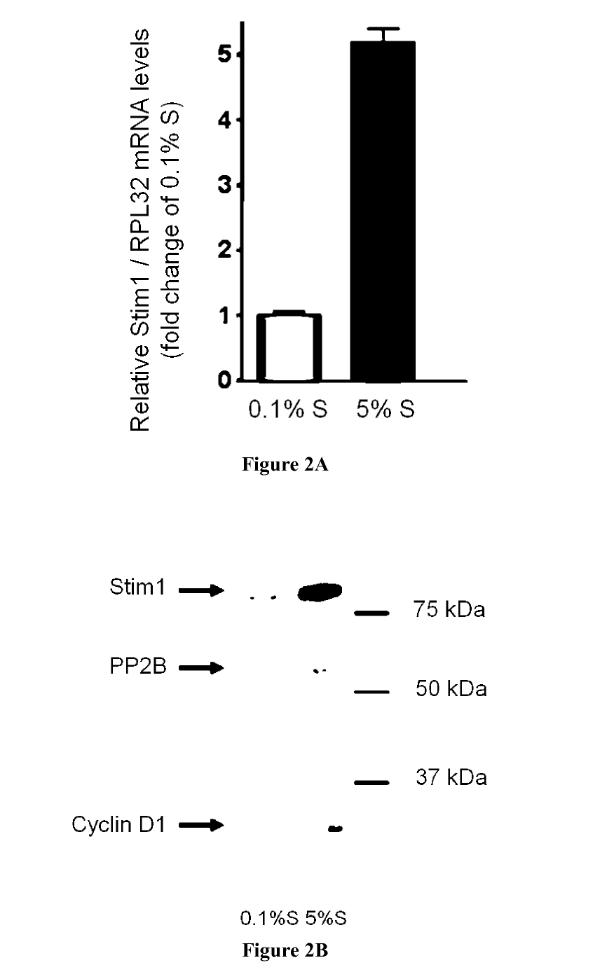

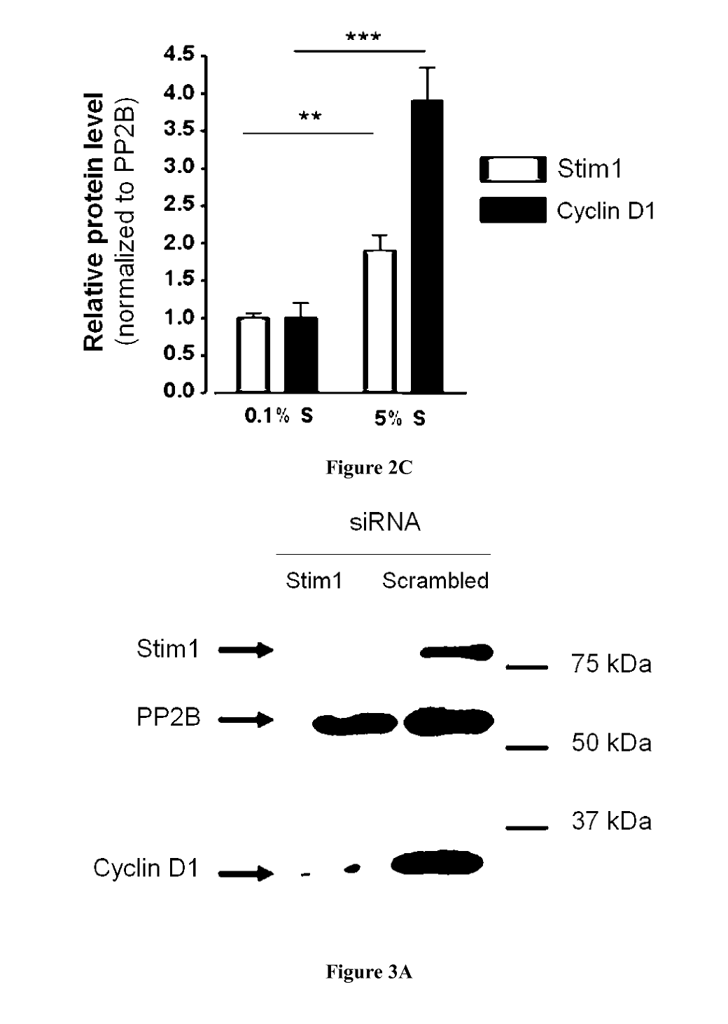

STIM1 is upregulated in proliferative VSMC: Relative expression level of STIM1 mRNA was obtained by quantitative Real Time PCR in quiescent (0.1% supplement mix, S, cultured hCASMC) and proliferative (5% S cultured hCASMC), showing a 5.2±0.3-fold upregulation in proliferative condition (FIG. 2A). Semi-quantitative evaluation of STIM1 protein level was obtained by integrated density analysis of immunoblotting, showing a 1.9±0.3-fold overexpression in proliferative condition (p<0.01), which correlated with the overexpression of the SMC proliferation marker cyclinD1 (FIGS. 2B and C).

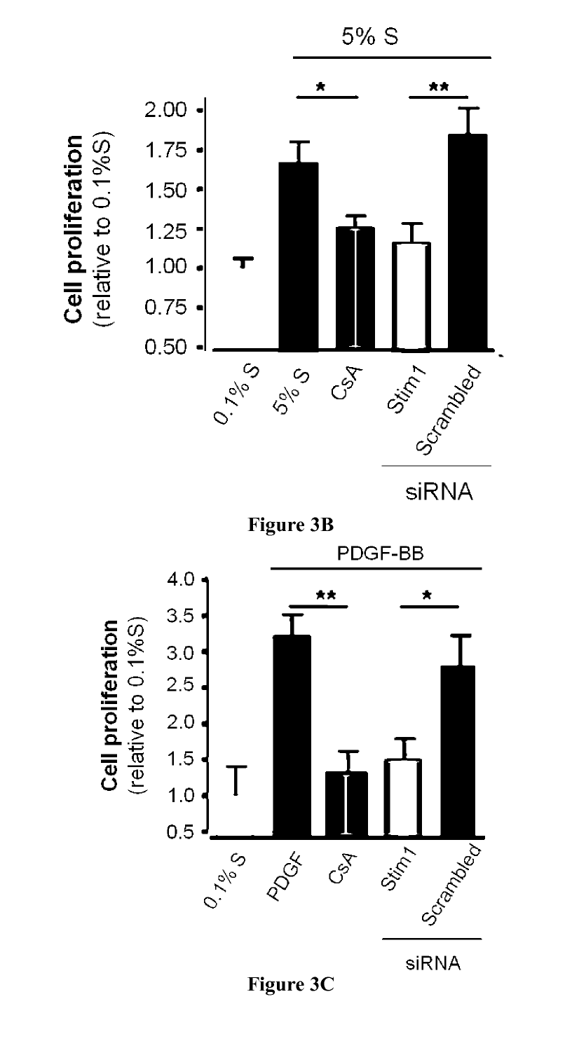

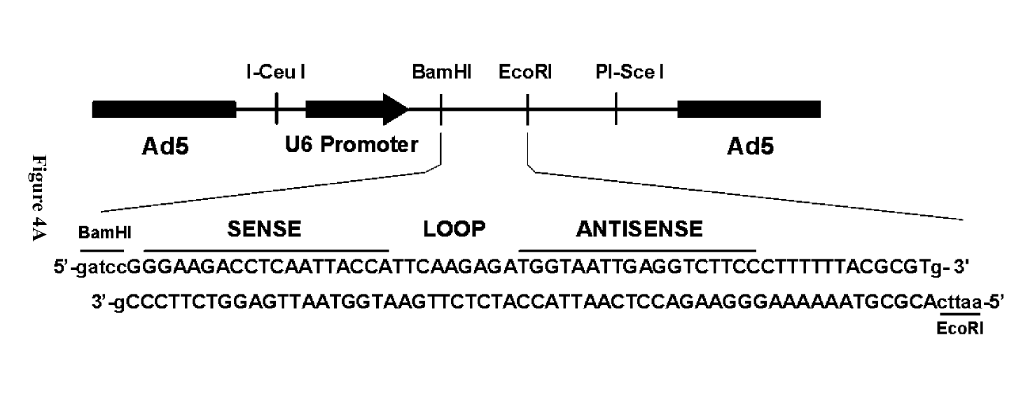

RNA interference-induced STIM1 silencing inhibits hCASMC proliferation in vitro: To further investigate the role of STIM1 in hCASMC proliferation, we used a RNAi based strategy to specifically silence STIM1 expression. Two siRNA common to human and rat STIM1 mRNA were designed: the sense sequence is 5′-GGGAAGACCUCAAUUACCAdtdt-3′ (SEQ ID NO:1) and anti-sense: 5′-UGGUAAUUGAGGUCUUCCCdtdt-3′ (SEQ ID NO:2). STIM1 siRNA transfection (50 nM) in cultured hCASMC induced a potent silencing of mRNA and protein: 72 hours after transfection, STIM1 mRNA was decreased by 91±3% and the protein by 95±4% (FIG. 3A) compared to scrambled siRNA transfected cells.

Supplement mix-induced proliferation was significantly lower in hCASMC transfected with STIM1 siRNA than in those transfected with scrambled siRNA (increase relative to 0.1% S: 116±12% and 184±16% respectively, p<0.01, FIG. 3B). Such inhibition was similar to the one observed with cyclosporine A, a classical calcineurin inhibitor. An identical pattern was observed when hCASMC were stimulated with the platelet derived growth factor (PDGF-BB), a more specific stimulator of NFAT-mediated signalling in VSMC (FIG. 3C). Similar results were obtained with alternatively designed and validated STIM1 siRNA. Finally, we observed that STIM1 silencing did not induce apoptosis of hCASMC (FIG. 3).

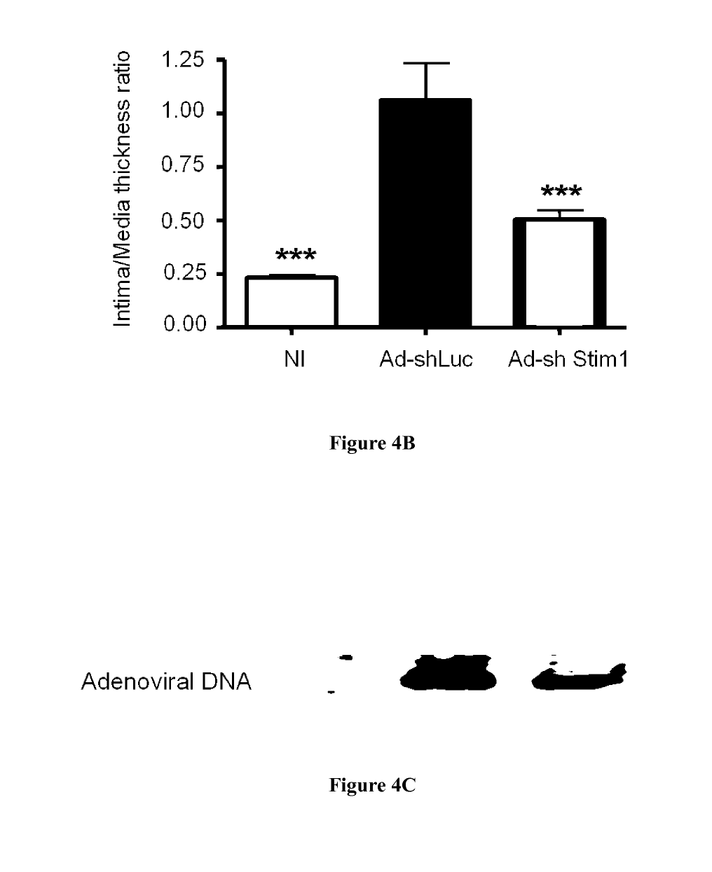

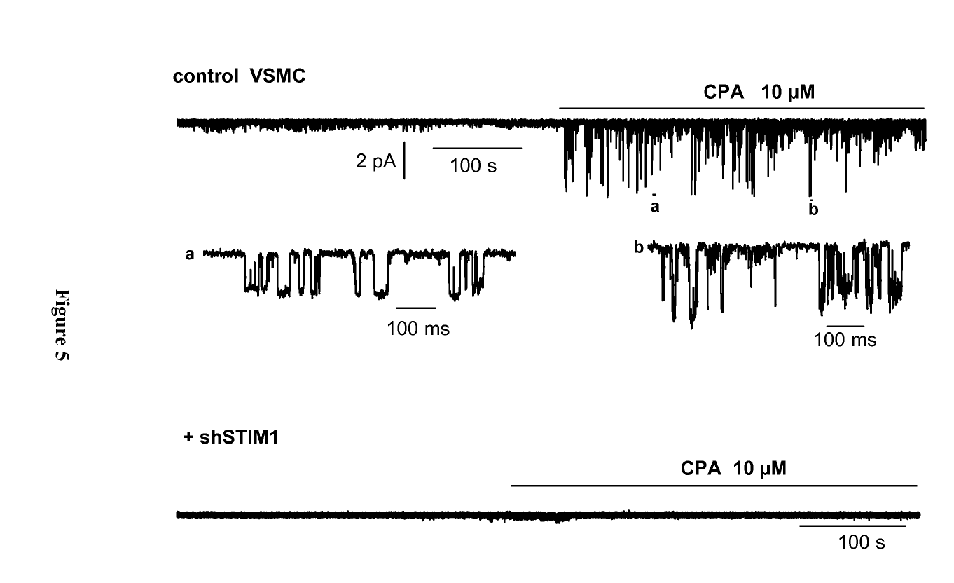

Adenoviral vector expressing specific STIM1 shRNA prevents in vivo neointima formation in rat injured carotid artery: To assess the role of STIM1 in preventing VSMC proliferation in vivo, we then infected balloon-injured rat carotid arteries with an adenoviral vector expressing a short hairpin RNA against rat STIM1 mRNA (Ad-shSTIM1, FIG. 4A). The shRNA comprises a sense sequence is 5′-GGGAAGACCTCAATTACCA-3′ (SEQ ID NO:3) and an anti-sense sequence 5′-TGGTAATTGAGGTCTTCCC-3′ (SEQ ID NO:4). The capacity of Ad-shSTIM1 to silence STIM1 expression was verified in vitro on rat arterial SMC. Seventy-two hours after infection, STIM1 mRNA and protein levels were lower than in cells infected with the same adenovirus expressing a luciferase shRNA (Ad-shLuc) (FIG. 4).

Two weeks after injury and infection with 10¹¹ DNA particles of either Ad-shSTIM1 or Ad shLuc, rats were sacrificed and morphometric analysis of injured carotids was performed on hematoxylin/eosin stained cross-sections. The degree of restenosis was determined by measuring intima and media thickness and by calculating the intima/media (I/M) thickness ratio. I/M ratios were significantly lower in Ad-shSTIM1-infected arteries than in Ad-shLuc-infected arteries (0.50±0.04 vs 1.06±0.17, p<0.0005, FIG. 4B). To confirm adenoviral infection, carotid DNA was extracted from each sample and adenovirus DNA was detected by PCR with specific primers (FIG. 4C). These results show that inhibition of STIM1 activity in turn inhibits VSMC proliferation in vitro and balloon injury-induced neointima formation in vivo.

STIM1 silencing inhibits TRPC currents: Channel activity was recorded for very long periods on membranes of CA VSMCs cultured in the presence of serum and growth factors and transfected with either scrambled siRNA or STIM1 siRNA (FIG. 5). The holding potential was maintained at −80 mV. Application of cyclopiazonic acid (CPA, 10 μM) induced a dramatic increase in spontaneously gating non-selective cation channels having a unitary channel conductance of different sates. CPA-induced channel activity was blocked in cells transfected with STIM1 siRNA.

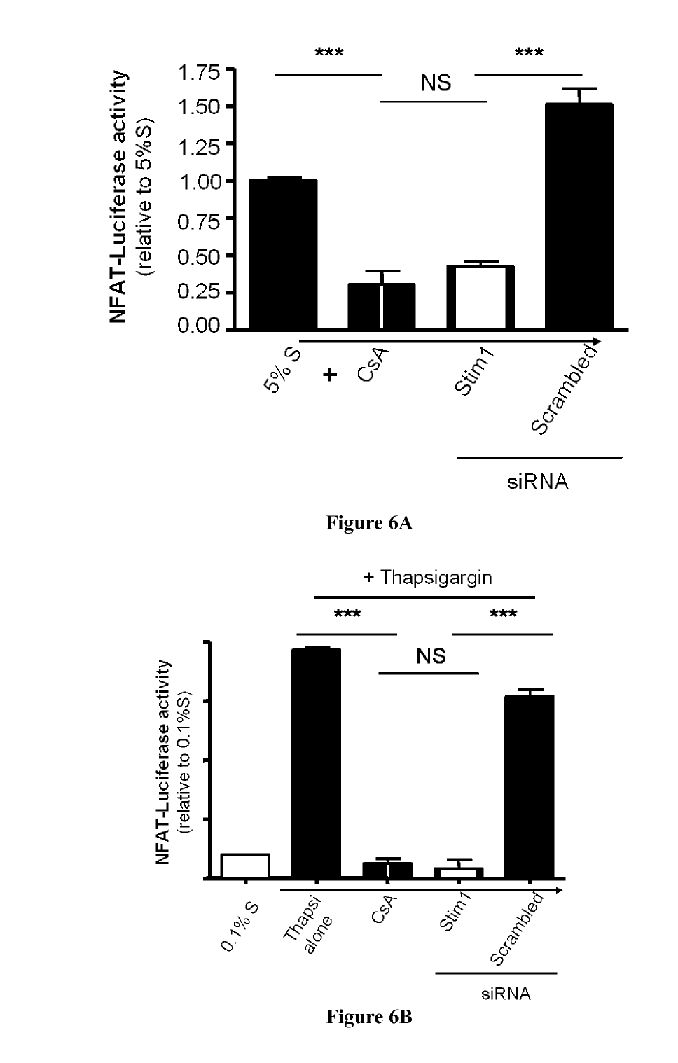

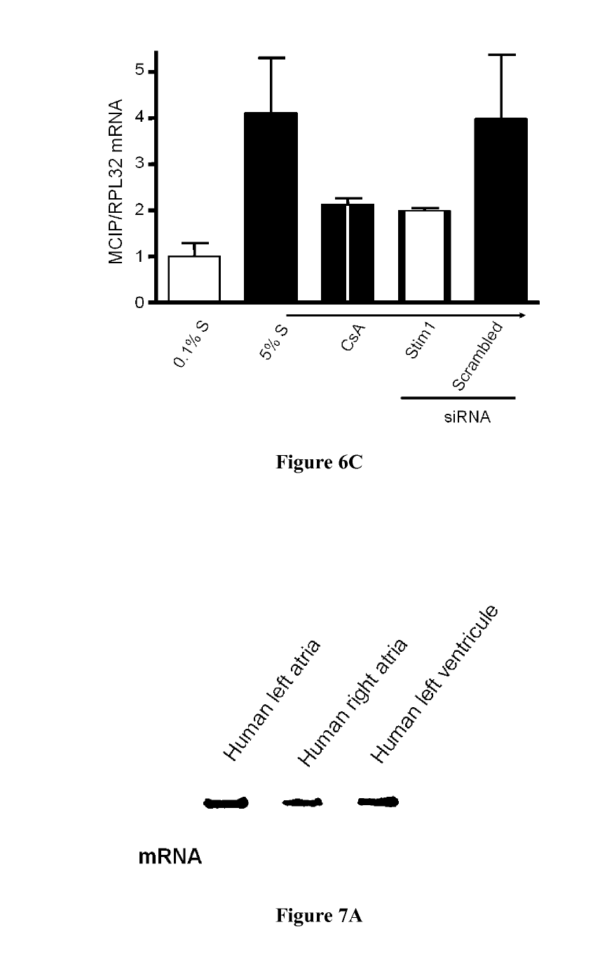

RNA interference-induced STIM1 silencing prevents NFAT nuclear translocation and activity and enhances CREB activity: In order to determine the pathway relating STIM1 to proliferation, we tested the activity of two Ca²⁺-regulated transcription factors: NFAT and CREB. NFAT activity was evaluated by measuring the activity of a NFAT-driven luciferase construct co-transfected with either shSTIM or scrambled siRNA, STIM1 siRNA transfected cells had a much lower luciferase activity than scrambled siRNA transfected cells (relative value of control hCASMC 5%: 42±4% vs 151±10%, p<0.001), comparable to that of CsA treated cells (30±9%, p=NS) (FIG. 6A). This effect was also observed in response to thapsigargin (TG), the TG-dependent activation of NFAT being drastically decreased in STIM1 siRNA transfected cells.

In control cells (5% S) as well as in scrambled siRNA-transfected cells, NFAT was mainly in the nucleus, whereas in STIM1 siRNA-transfected cells NFAT was in the cytosol. Finally we measured the expression of MCIP1 (modulatory calcineurin interacting protein1) a gene driven by NFAT. MCIP1 mRNA was increased in presence of growth supplement (5% S). Inhibition of calcineurin by CsA prevented MCIP1 expression, as expected. MCIP1 mRNA level was much lower in STIM1 siRNA-transfected cells than in scrambled siRNA-transfected cells. Together these data indicate that silencing STIM results in NFAT inactivation.

The influence of STIM1 on the activity of CRE was measured using a CRE-luciferase construct. The activity of CRE was higher in cells transfected with STIM1 siRNA than in cells transfected with scrambled_siRNA (FIG. 6C).

### EXAMPLE 2

### STIM1 and Cardiomyocyte Hypertrophy

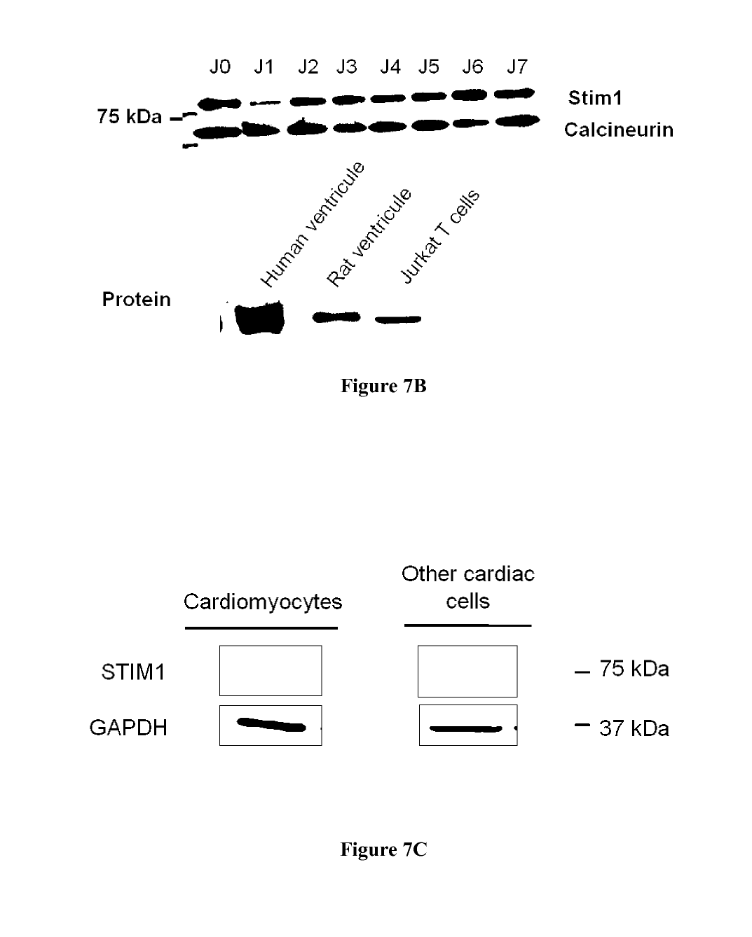

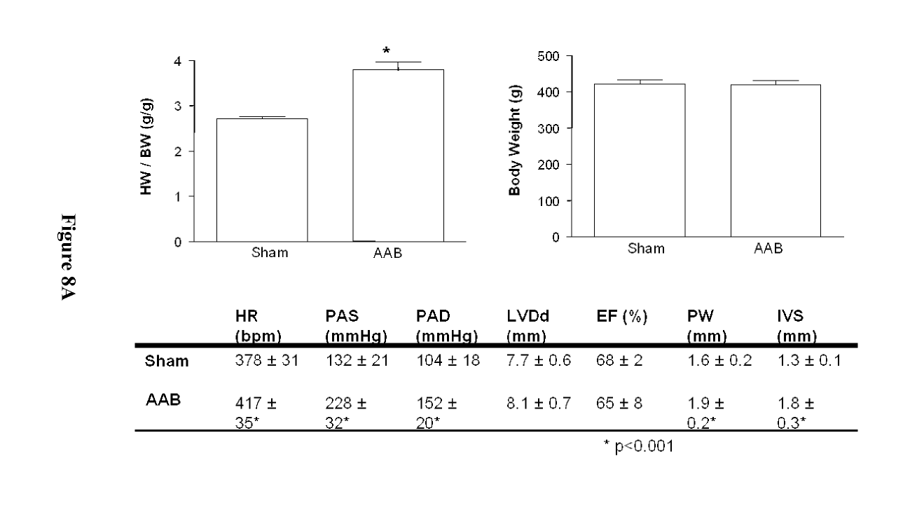

STIM1 mRNA was detected by PCR in the human heart, in both atria and ventricles (FIG. 7A). The protein was also detected in human and rat ventricles (FIG. 7B). STIM1 protein was detected by Western-blot and immunofluorescence in isolated adult or neonatal cardiomyocytes (FIG. 7B). Stim1 expression persist for at least 7 days in culture (FIG. 7B).

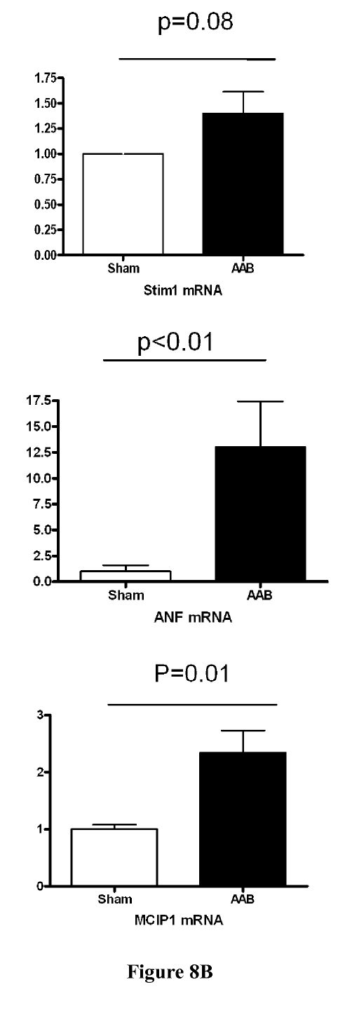

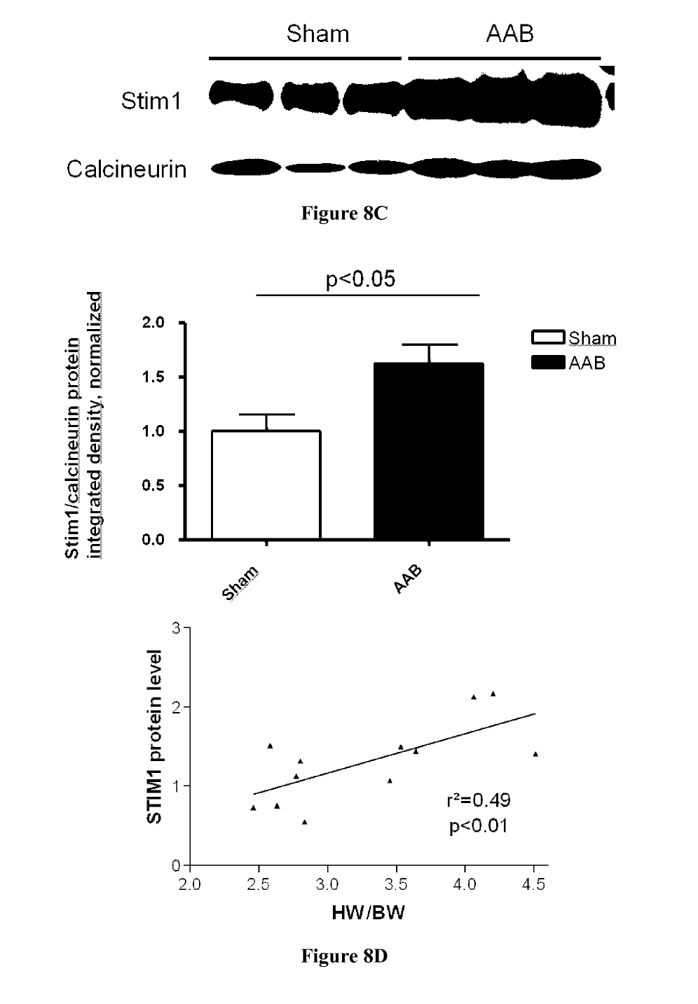

To determine the expression of STIM1 in pathological growth, we used a model of pressure overload induced by abdominal aortic banding (AAB) in the rat. As shown in FIG. 8A the heart weight/to body weight ratio was increased in the AAB without increase in total body weight reflecting pathological cardiac growth. This pathological cardiac growth was confirmed by an increased ANF and MCIP mRNA levels, two markers of cardiac hypertrophy (FIG. 8B). Interestingly, STIM1 mRNA level detected by qRT-PCR was also significantly increased (p=0.08) (FIG. 8B). The expression in STIM1 expression was confirmed at the protein level by western-blotting (FIG. 8C) and the increase in STIM1 protein level was correlated to the increase in HW/BW and ratio (FIG. 8D).

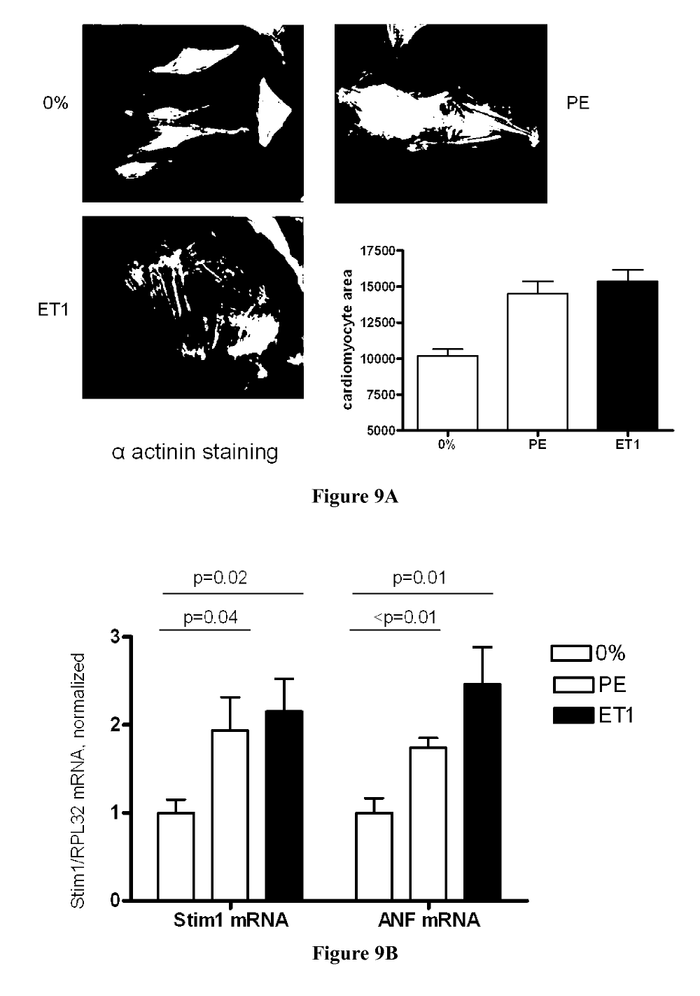

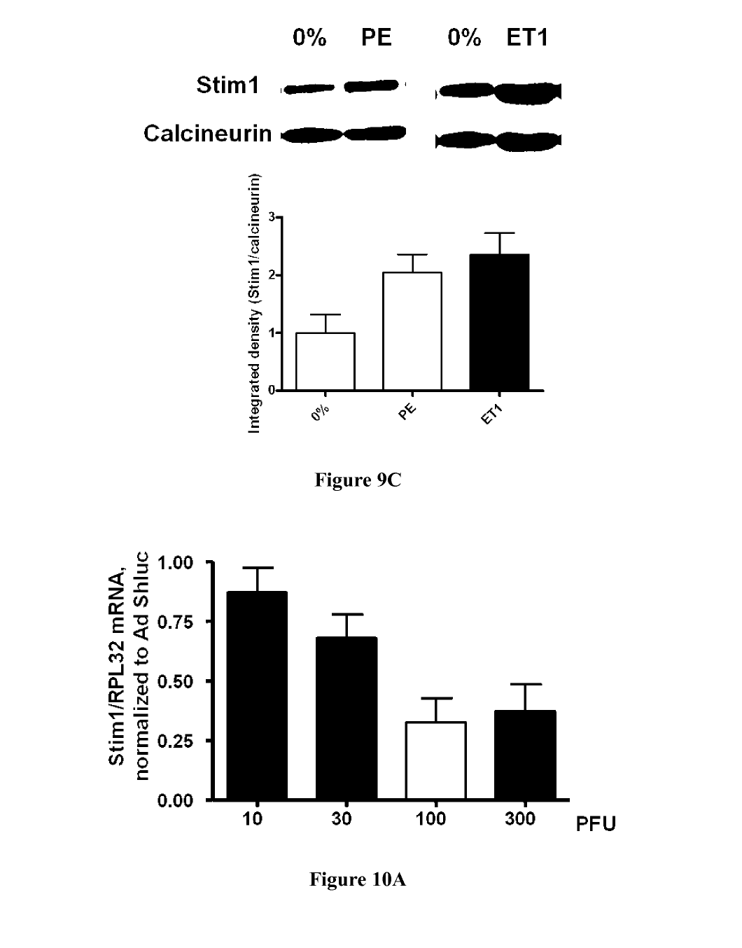

To confirm overexpression of STIM1 in pathological growth we used an in vitro of neonatal cardiomyocytes stimulated with growth stimuli such as endothelin 1 (ET1) or phenylephrine (PE). The efficiency of PE and ET1 to induce growth was analyzed by measuring the area of the cardiomyocyte after immunolabelling with anti-beta-actinin antibody. As shown in FIG. 9A, treatment with PE (50 μM) and ET1 (1 μM) for 48 hours induced cardiac growth. In these conditions, STIM1 as well as ANF mRNA levels, used as a control and measured by QRT-PCR were significantly increased (FIG. 9B). The level of STIM1 protein, normalized to calcineurin (PP2B) level, was also increased by ET1 and PE (FIG. 9C).

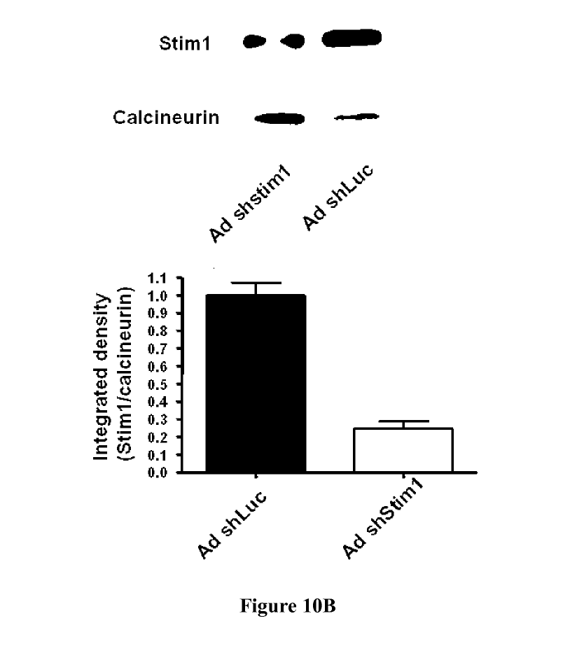

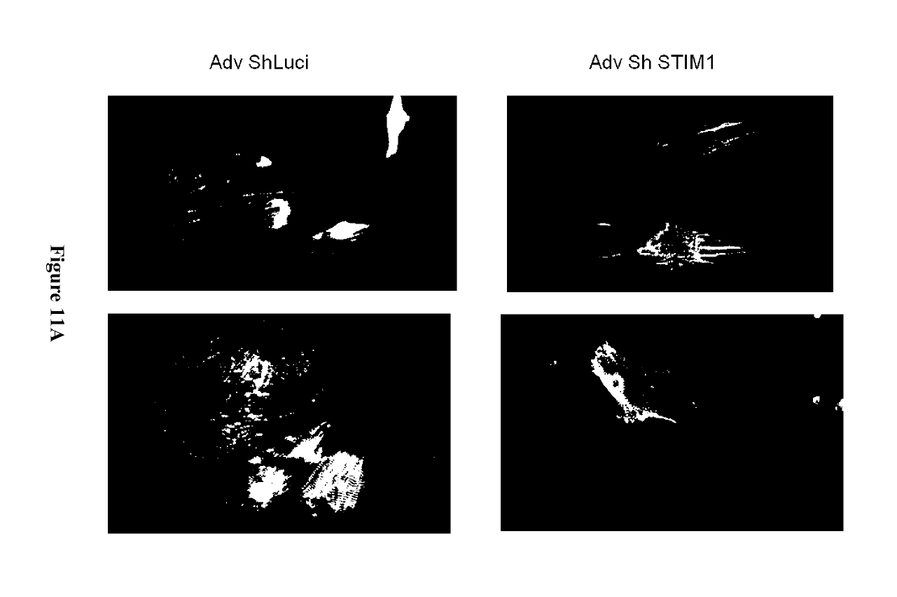

Inhibition of STIM1 was obtained using an adenoviral vector encoding a STIM1 shRNA (the same as Example 1) and was compared to a negative control encoding sh luciferase (Ad shLuc). As shown in FIG. 10A, STIM1 mRNA normalized to RPL32 mRNA, was 70% lower in neonatal cardiomyocytes infected with 100 PFU of Ad shSTIM when compared to cardiomyocytes infected with Ad shLuc. At this dose, the protein level was decreased by about 80% in neonatal cardiomyocytes infected with ad ShSTIM1 compared to cardiomyocytes infected with Ad sh Luc (FIG. 10B).

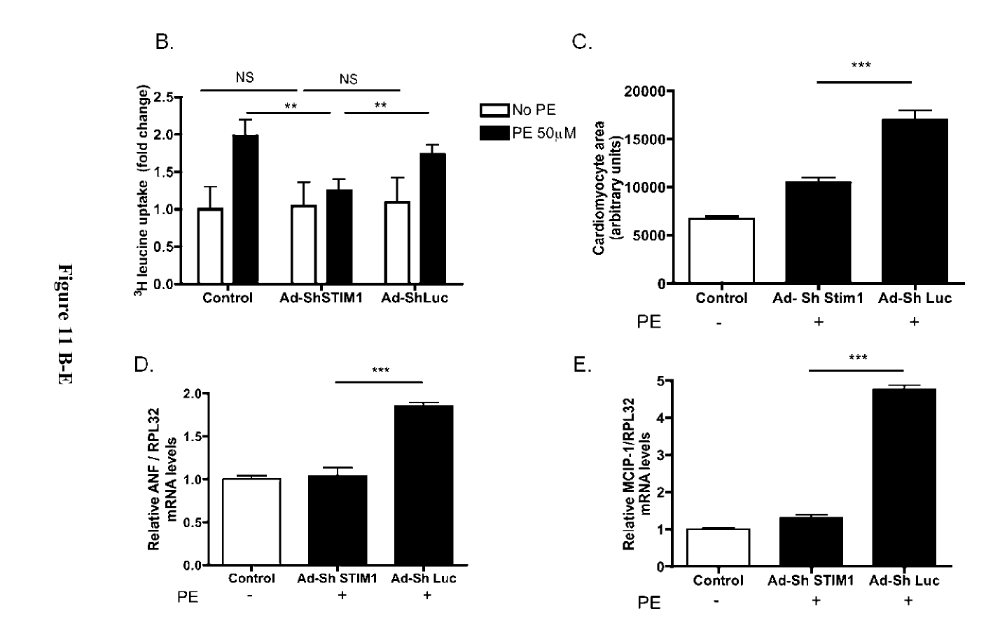

Neonatal cardiomyocytes were infected with either Ad sh STIM1 or with Ad shLuc for 2 days and then stimulated with PE (50 μM) for 2 days. They were then fixed and labelled with anti-beta-actinin (FIG. 11A). In cardiomyocytes infected with Ad shLuc and treated with PE the myocytes area was greater than in control cell non-infected and not treated with PE as expected. Ad sh STIM prevented PE-induced cardiomyocyte hypertrophy (FIG. 11B) and also prevented PE increased in ANF and MCIP expression (FIG. 11C).

### REFERENCES

Throughout this application, various references describe the state of the art to which this invention pertains. The disclosures of these references are hereby incorporated by reference into the present disclosure.
Brummelkamp T R, Bernards R, Agami R. A system for stable expression of short interfering RNAs in mammalian cells. Science. 2002 Apr. 19; 296(5567):550-3.
Choi V W, Samulski R J, McCarty D M. Effects of adeno-associated virus DNA hairpin structure on recombination. J. Virol. 2005 June; 79(11):6801-7.
Lipskaia L, del Monte F, Capiod T, Yacoubi S, Hadri L, Hours M, Hajjar R J, Lompré A M. Sarco/endoplasmic reticulum Ca2+-ATPase gene transfer reduces vascular smooth muscle cell proliferation and neointima formation in the rat. Circ Res. 2005 Sep. 2; 97(5):488-95. Epub 2005 Aug. 4.
Colas P, Cohen B, Jessen T, Grishina I, McCoy J, Brent R. (1996) Genetic selection of peptide aptamers that recognize and inhibit cyclin-dependent kinase 2. Nature, 380, 548-50.
Cole et al., Monoclonal Antibodies and Cancer Therapy, Alan R. Liss, Inc., 1985, pp. 77-96).
Cote R J, Morrissey D M, Houghton A N, Beattie E J Jr, Oettgen H F, Old L J. Generation of human monoclonal antibodies reactive with cellular antigens. Proc Natl Acad Sci USA. 1983 April; 80(7):2026-30.
Dzau V J, Braun-Dullaeus R C, Sedding D G. Vascular proliferation and atherosclerosis: new perspectives and therapeutic strategies. Nat. Med. 2002 November; 8(11):1249-56.
Elbashir S M, Harborth J, Lendeckel W, Yalcin A, Weber K, Tuschl T. Duplexes of 21-nucleotide RNAs mediate RNA interference in cultured mammalian cells. Nature. 2001 May 24; 411(6836):494-8.
Hannon G J. RNA interference. Nature. 2002 Jul. 11; 418(6894):244-51.
Jayasena S. D. (1999) Aptamers: an emerging class of molecules that rival antibodies in diagnostics. Clin Chem. 45(9):1628-50.
Kohler G, Milstein C. Continuous cultures of fused cells secreting antibody of predefined specificity. Nature. 1975 Aug. 7; 256(5517):495-7.
Kriegler, A Laboratory Manual,” W. H. Freeman C.O., New York, 1990.
McManus M T, Sharp P A. Gene silencing in mammals by small interfering RNAs. Nat Rev Genet. 2002 October; 3(10):737-47.
Murry, “Methods in Molecular Biology,” vol. 7, Humana Press, Inc., Cliffton, N.J., 1991.
Novak K. Cardiovascular disease increasing in developing countries. Nat. Med. 1998 September; 4(9):989-90.
Tuerk C. and Gold L. (1990) Systematic evolution of ligands by exponential enrichment: RNA ligands to bacteriophage T4 DNA polymerase. Science. 3; 249(4968):505-10.
Tuschl T, Zamore P D, Lehmann R, Bartel D P, Sharp P A. Targeted mRNA degradation by double-stranded RNA in vitro. Genes Dev. 1999 Dec. 15; 13(24):3191-7.
Wu Z, Asokan A, Samulski R J. Adeno-associated virus serotypes: vector toolkit for human gene therapy. Mol. Ther. 2006 September; 14(3):316-27. Epub 2006 Jul. 7.

1-10. (canceled)

11. A method for treating or preventing a cardiovascular disorder a patient in need thereof, which method comprises administering an inhibitor of STIM1 to said patient.

12. A method for inhibiting the proliferation or growth of smooth muscle cells or cardiomyocytes which method comprises administering an inhibitor of STIM1 to said patient.

13. The method according to claim 11 wherein the cardiovascular disorder is selected from the group consisting of atherosclerosis, post-angioplasty restenosis, pulmonary arterial hypertension, vein-graft disease, cardiac hypertrophy, cardiac arrhythmias, valvulopathies, diastolic dysfunction, chronic heart failure, ischemic heart failure, and myocarditis.

14. The method according to claim 11, wherein the inhibitor is an inhibitor of STIM1 expression.

15. The method according to claim 14, wherein said inhibitor of STIM1 expression is selected from the group consisting of antisense RNA or DNA molecules, small inhibitory RNAs (siRNAs), short hairpin RNA and ribozymes.

16. The method according to claim 11, wherein said inhibitor of STIM1 is selected from the group consisting of small organic molecules, aptamers antibodies and antibody fragments.

17. A pharmaceutical composition for inhibiting the proliferation of smooth muscle cells or the hypertrophic response of cardiomyocytes, comprising an inhibitor of STIM1.

18. A pharmaceutical composition for treating a cardiovascular disorder, comprising an inhibitor of STIM1.

19. A biomaterial or medical delivery device comprising an inhibitor of STIM1.

20. A biomaterial or medical delivery device according to claim 19, wherein said biomaterial or medical delivery device is selected in the group consisting of a stent, a bypass graft, an internal patch around the vascular tube, an external patch around the vascular tube, a vascular cuff and a angioplasty catheter.


SEQUENCE LISTING (4 sequences)

SEQ ID NO: 1 - RNA, 19 nt, Artificial
  siRNA
gggaagaccucaauuacca

SEQ ID NO: 2 - RNA, 19 nt, Artificial
  siRNA
ugguaauugaggucuuccc

SEQ ID NO: 3 - DNA, 19 bp, Artificial
  shRNA
gggaagacctcaattacca

SEQ ID NO: 4 - DNA, 19 bp, Artificial
  shRNA
tggtaattgaggtcttccc